(19) United States
(12) Patent Application Publication
(10) Pub. No.: US 2011/0118283 A1
(43) Pub. Date: May 19, 2011

(54) Substituted Pyrrolidine-2-Carboxamides

(76) Inventors: Qingjie Ding, Bridgewater, NJ (US); Nan Jiang, Fairfield, NJ (US); Jin-Jun Liu, Warren Township, NJ (US); Jing Zhang, Parsippany, NJ (US); Zhuming Zhang, Hillsborough, NJ (US)

(21) Appl. No.: 12/898,955

(22) Filed: Oct. 6, 2010

Related U.S. Application Data
(60) Provisional application No. 61/261,913, filed on Nov. 17, 2009.

(51) Int. Cl. A61K 31/506; C07D 409/14; C07D 409/04; C07D 401/14; C07D 401/04; C07D 405/14; C07D 403/04; A61K 31/4025; A61K 31/4439; A61P 35/00

(52) U.S. Cl. 514/256; 548/527; 546/279.1; 544/334; 514/422; 514/343

(57) ABSTRACT

There are provided compounds of the formula

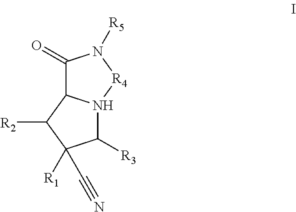

wherein R₁, R₂, R₃, R₃, R₄, R₅ are as described herein
and enantiomers, pharmaceutically acceptable salts and esters thereof. The compounds are useful as anticancer agents.

### PRIORITY TO RELATED APPLICATION(S)

This application claims the benefit of U.S. Provisional Application No. 61/261,913, filed Nov. 17, 2009, which is hereby incorporated by reference in its entirety.

### SUMMARY OF THE INVENTION

The present invention relates to pyrrolidine-2-carboxamide derivatives I which act as antagonists of mdm2 interactions and hence are useful as potent and selective anticancer agents. The present compounds are of the general formula

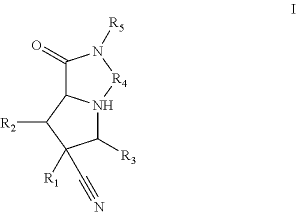

wherein R₁, R₂, R₃, R₃, R₄, R₅ are as described herein
and enantiomers and pharmaceutically acceptable salts and esters thereof.

### BACKGROUND OF THE INVENTION

p53 is a tumor suppresser protein that plays a central role in protection against development of cancer. It guards cellular integrity and prevents the propagation of permanently damaged clones of cells by the induction of growth arrest or apoptosis. At the molecular level, p53 is a transcription factor that can activate a panel of genes implicated in the regulation of cell cycle and apoptosis. p53 is a potent cell cycle inhibitor which is tightly regulated by MDM2 at the cellular level. MDM2 and p53 form a feedback control loop. MDM2 can bind p53 and inhibit its ability to transactivate p53-regulated genes. In addition, MDM2 mediates the ubiquitin-dependent degradation of p53. p53 can activate the expression of the MDM2 gene, thus raising the cellular level of MDM2 protein. This feedback control loop insures that both MDM2 and p53 are kept at a low level in normal proliferating cells. MDM2 is also a cofactor for E2F, which plays a central role in cell cycle regulation.

The ratio of MDM2 to p53 (E2F) is dysregulated in many cancers. Frequently occurring molecular defects in the p16INK4/p19ARF locus, for instance, have been shown to affect MDM2 protein degradation. Inhibition of MDM2-p53 interaction in tumor cells with wild-type p53 should lead to accumulation of p53, cell cycle arrest and/or apoptosis. MDM2 antagonists, therefore, can offer a novel approach to cancer therapy as single agents or in combination with a broad spectrum of other antitumor therapies. The feasibility of this strategy has been shown by the use of different macromolecular tools for inhibition of MDM2-p53 interaction (e.g. antibodies, antisense oligonucleotides, peptides). MDM2 also binds E2F through a conserved binding region as p53 and activates E2F-dependent transcription of cyclin A, suggesting that MDM2 antagonists might have effects in p53 mutant cells.

### DETAILED DESCRIPTION OF THE INVENTION

The present invention relates to pyrrolidine-2-carboxamide derivatives I which act as antagonists of mdm2 interactions and hence are useful as potent and selective anticancer agents. The present compounds are of the general formula

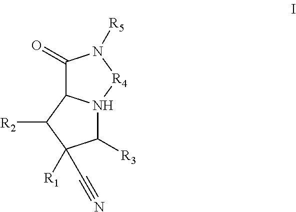

wherein
R₁ is a substituted or unsubstituted heteroaryl selected from

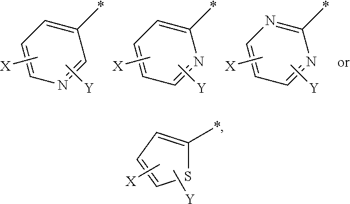

X is selected from the group consisting of H, F, Cl, Br and I,
Y is H or F,
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄ and R₅ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂C^{H}₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle, or substituted hetereocycle,
and in the case of R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle,
m, n and p are independently 0 to 6
   and the pharmaceutically acceptable salts and esters thereof.

Preferred above are compounds of formula I wherein R₂ is a substituted aryl, i.e., a substituted phenyl selected from

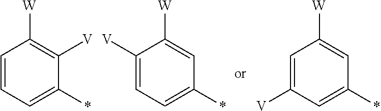

wherein
W is F, Cl or Br,
V is H or F,
R₃ is a substituted lower alkyl selected from

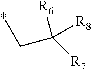

where R₆, R₇ are both methyl, or linked to form a cyclopropyl, cyclobutyl, cyclopentyl or cyclohexyl group,
R₈ is (CH₂)_{q}—R₉
q is 0, 1 or 2,
   and
R₉ is selected from hydrogen, hydroxyl, lower alkyl, lower alkoxy, aryl, substituted aryl. hetereoaryl, substituted heteroaryl, hetereocycle or substituted heterocycle.

Preferred are compounds of formula I having a stereochemical structure as shown as formula II

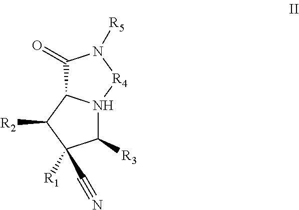

wherein
R₁ is a substituted or unsubstituted heteroaryl selected from

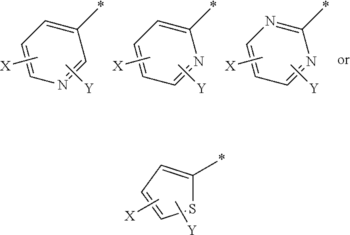

X is selected from the group consisting of H, F, Cl, Br and I
Y is H or F
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄and R₅ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, —
R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle, or substituted hetereocycle,
and in the case of R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle,
m, n and p are independently 0 to 6
   and the pharmaceutically acceptable salts and esters thereof.

Preferred above are compounds of formula H wherein R₂ is a substituted phenyl selected from

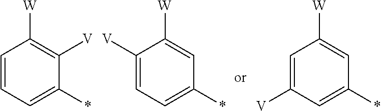

W is F, Cl or Br,
V is H or F,
and R₃ is a substituted lower alkyl selected from

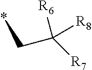

where R_{6,} R₇ are both methyl, or linked to form a cyclopropyl, cyclobutyl, cyclopentyl or acyclohexyl group,
R₈ is (CH₂)_{q}—R₉
q is 0, 1 or 2
   and
R₉ is selected from hydrogen, hydroxyl, lower alkyl, lower alkoxy, aryl, substituted aryl. hetereoaryl, substituted heteroaryl, hetereocycle or substituted heterocycle.

Further preferred are compounds in formula II wherein
R₁ is a substituted or unsubstituted heteroaryl selected from

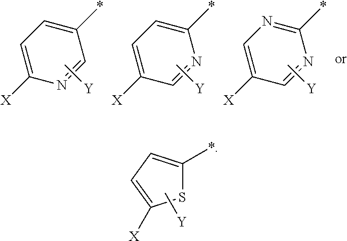

X is selected from the group consisting of F, Cl, Br,
Y is H or F,
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄ is hydrogen and R₅ is (CH₂)ₙ—R′,
n is 0 or 1 and
R′ is selected from aryl, substituted aryl, hetereoaryl, substituted heteroaryl, hetereocycle or substituted heterocycle
   and the pharmaceutically acceptable salts and esters thereof.

More preferred of the above formula II compounds are those wherein R₂ is a substituted aryl, i.e., a substituted phenyl selected from

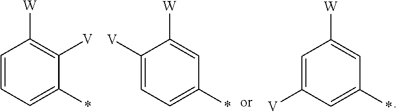

W is F, Cl or Br,
V is H or F,
and R₃ is a substituted lower alkyl selected from

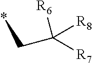

where R₆, R₇ are both methyl, or linked to form a cyclopropyl, cyclobutyl, cyclopentyl or acyclohexyl group,
R₈ is (CH₂)_{q}—R₉
q is 0, 1 or 2
   and
R₉ is selected from hydrogen, hydroxyl, lower alkyl, lower alkoxy, aryl, substituted aryl. hetereoaryl, substituted heteroaryl, hetereocycle or substituted heterocycle.

Especially preferred are compounds selected from the group consisting of
rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid ((S)-3,4-dihydroxy-butyl)-amide,
rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide,
rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide,
chiral-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide,
rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid ((S)-3,4-dihydroxy-butyl)-amide,
rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide,
rac-4-{[(2R,3S,4S,5S)-3-(3-chloro-2-fluoro-phenyl)-4-(5-chloro-pyridin-2yl)-4-cyano-5-(2,2-dimethyl-propyl)-pyrrolidine-2-carbonyl]-amino}-benzoic acid methyl ester,
rac-4-{[(2R,3S,4S,5S)-3-(3-chloro-2-fluoro-phenyl)-4-(5-chloro-pyridin-2yl)-4-cyano-5-(2,2-dimethyl-propyl)-pyrrolidine-2-carbonyl]-amino}-benzoic acid,
rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid methyl ester,
rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid,
rac-(2R,3R,4R,5S)-3-(3-chloro-phenyl)-4-cyano-5-(2,2-dimethyl-propyl)-4-pyridin-3-yl-pyrrolidine-2-carboxylic acid ((S)-3,4-dihydroxy-butyl)-amide,
rac-(2R,3R,4R,5S)-3-(3-chloro-phenyl)-4-(6-chloro-pyridin-3-yl)-4-cyano-5-(2,2-dimethyl-propyl-pyrrolidine-2-carboxylic acid [2-((S)-22-dimethyl-[1,3]dioxolan-4-yl)-ethyl]-amide,
rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid ((S)-3,4-dihydroxy-butyl)-amide,
rac-4-{[(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carbonyl]-amino}-3-methoxy-benzoic acid methyl ester and
rac-4-{[(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carbonyl]-amino}-3-fluoro-benzoic acid methyl ester.

### Terms & Definitions

In the specification where indicated the various groups may be substituted by 1-5 or, preferably, 1-3 substituents independently selected from the group consisting of lower alkyl, lower-alkenyl, lower-alkynyl, dioxo-lower-alkylene (forming e.g. a benzodioxyl group), halogen, hydroxy, CN, CF₃, NH₂, N(H, lower-alkyl), N(lower-alkyl)₂, aminocarbonyl, carboxy, NO₂, lower-alkoxy, thio-lower-alkoxy, lower-alkylsufonyl, aminosulfonyl, lower-alkylcarbonyl, lower-alkylcarbonyloxy, lower-alkoxycarbonyl, lower-alkyl-carbonyl-NH, fluoro-lower-alkyl, fluoro-lower-alkoxy, lower-alkoxy-carbonyl-lower-alkoxy, carboxy-lower-alkoxy, carbamoyl-lower-alkoxy, hydroxy-lower-alkoxy, NH₂-lower-alkoxy, N(H, lower-alkyl)-lower-alkoxy, N(lower-alkyl)₂-lower-alkoxy, lower-alkyl-1-oxiranyl-lower-alkoxy-lower-alkyl, 2-oxo-pyrrolidin-1-yl, (1,1-dioxo)-2-isothiazolidine, 3-lower-alkyl sulfinyl, a substituted or unsubstituted heterocyclic ring, a substituted or unsubstituted aryl ring, a substituted or unsubstituted heteroaryl ring, trifluoro-lower-alkylsulfonylamino-aryl, lower-alkyl sulfonylaminocarbonyl, lower-alkyl sulfonylaminocarbonyl-aryl, hydroxycarbamoyl-phenyl, benzyloxy-lower-alkoxy, mono- or di-lower alkyl substituted amino-sulfonyl and lower-alkyl which can optionally be substituted with halogen, hydroxy, NH₂, N(H, lower-alkyl) or N(lower-alkyl)₂. Preferred substituents for the cycloalkyl, cycloalkenyl, aryl, heteroaryl and heterocycle rings are halogen, lower alkoxy, lower alkyl, hydroxycarbonyl, carboxy, carboxy lower alkoxy, oxo and CN. Preferred substituents for alkyl are alkoxy and N(lower alkyl)₂.

The term “alkyl” refers to straight- or branched-chain saturated hydrocarbon groups having from 1 to about 20 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, and in certain embodiments from 1 to 4 carbon atoms. Examples of alkyl groups include, but are not limited to, methyl, ethyl, n-propyl, i-propyl, n-butyl, s-butyl, t-butyl, n-pentyl, and s-pentyl.

As used herein, “cycloalkyl” is intended to refer to any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or Spiro compounds. Examples of cycloalkenyls include, but are not limited to, cyclopentenyl or cyclohexenyl.

The term “alkenyl” as used herein means an unsaturated straight-chain or branched aliphatic hydrocarbon group containing one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “halogen” as used in the definitions means fluorine, chlorine, bromine, or iodine, preferably, fluorine and chlorine.

“Aryl” means a monovalent, monocyclic or bicyclic, aromatic carbocyclic hydrocarbon radical, preferably a 6-10 member aromatic ring system. Preferred aryl groups include, but are not limited to, phenyl, naphthyl, tolyl, and xylyl.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole and tetrazolyl.

In the case of aryl or heteroaryl which are bicyclic it should be understood that one ring may be aryl while the other is heteroaryl and both may be substituted or unsubstituted.

“Heterocycle” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like.

“Hetero atom” means an atom selected from N, O and S.

“Alkoxy, alkoxyl or lower alkoxy” refers to any of the above lower alkyl groups attached to an oxygen atom. Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

“Pharmaceutically acceptable,” such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like.

Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (6th Ed. 1995) at pp. 196 and 1456-1457.

The compounds of formula I and II as well as their salts that have at least one asymmetric carbon atom may be present as racemic mixtures or different stereoisomers. The various isomers can be isolated by known separation methods, e.g., chromatography. Compounds disclosed herein and covered by formula I and II above may exhibit tautomerism or structural isomerism. It is intended that the invention encompasses any tautomeric or structural isomeric form of these compounds, or mixtures of such forms, and is not limited to any one tautomeric or structural isomeric form depicted in the formulas above.

The compounds of the present invention are useful in the treatment or control of cell proliferative disorders, in particular oncological disorders. These compounds and formulations containing said compounds may be particularly useful in the treatment or control of solid tumors, such as, for example, breast, colon, lung and prostate tumors.

A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration; it may be given as continuous infusion.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a formula I compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

“Effective amount” means an amount that is effective to prevent, alleviate or ameliorate symptoms of disease or prolong the survival of the subject being treated.

“IC50” refers to the concentration of a particular compound required to inhibit 50% of a specific measured activity. IC₅₀ can be measured, inter alia, as is described subsequently.

### Synthetic Methods

The present invention provides methods for the synthesis of substituted pyrrolidine-2-carboxamide. The compounds of the invention can be prepared by processes known in the art. Suitable processes for synthesizing these compounds are provided in the examples.

Compounds of this invention can be synthesized according to the following general schemes. The key transformation is a convergent [2+3] cylcoaddition of emine II and activated olefin III to generate pyrrolidine-3-carbonitrile compounds IV in a stereoselective and efficient manner.

The starting materials are either commercially available or can be synthesized by methods known to those of ordinary skill in the art. Preparations of intermediates II and III are illustrated in Scheme 1 and 2. In general an appropriately selected aldehyde can be reacted with glycine tert-butyl ester or glycine methyl ester to generate imine II and were used as a crude product (Scheme 1).

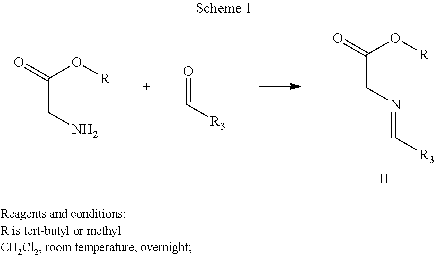

An intermediate of formula III can be made from a base-catalyzed condensation reaction of appropriately selected substituted-phenyl acetonitrile and aldehyde The reaction proceeds in a highly stereoselective manner with Z-isomer as the major or exclusive product.

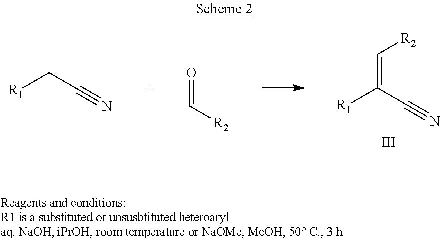

As illustrated in Scheme 3, pyrrolidine of formula IV can be made from intermediates II and III by a convergent 1,3-dipolar cylcoaddition reaction mediated by lewis acid AgF and triethylamine. The [2+3] cycloaddition reactions of azomethine ylides 1,3-dipoles with olefinic dipolarphiles to form pyrrolidine ring formation have been described in published procedures including Jorgensen, K. A. et al (Org. Lett. 2005, Vol 7, No. 21, 4569-4572), Grigg, R. et al (Tetrahedron, 1992, Vol 48, No. 47, 10431-10442; Tetrahedron, 2002, Vol 58, 1719-1737), Schreiber, S. L. et al (J. Am. Chem. Soc., 2003, 125, 10174-10175), and Carretero, J. C. et al (Tetrahedron, 2007, 63, 6587-6602). Compounds IV is subsequently converted to acid V followed by amide formation with various amines using HATU as the coupling reagent to give the compounds of formula I. The amide formation from V to I can also be achieved under other conditions using EDCI and HOBt, or oxalyl chloride, or diphenylphosphinic chloride as the coupling reagent to activate the acid V.

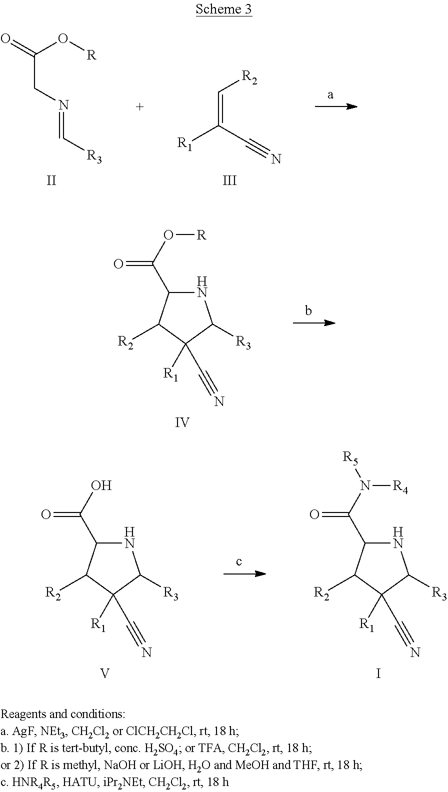

The pyrrolidine compounds I, IV, V are prepared initially as a racemic mixture and can be chirally separated using chiral Super Fluid Chromatography (SFC) or chiral HPLC or chiral column chromatography. For example, racemic mixture of compound Ia and Ia′ can be readily resolved into two optically pure or enriched chiral enantiomers by separation using chiral Super Fluid Chromatography (SFC). (Scheme 4).

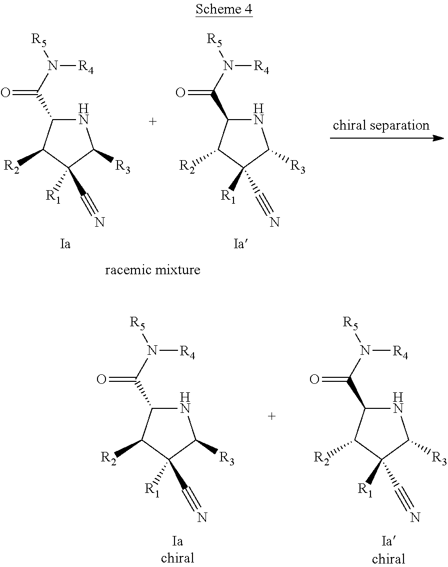

### EXAMPLES

The compounds of the present invention may be synthesized according to known techniques. The following examples and references are provided to aid the understanding of the present invention, the true scope of which is set forth in the appended claims.

### Example 1

### Preparation of intermediate [3,3-dimethyl-but-(E)-ylideneamino]-acetic acid tert-butyl ester

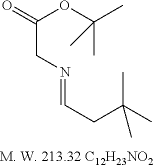

A mixture of glycine tert-butyl ester (Alfa) (2.71 g, 20.0 mmol) and 3,3-dimethyl-butyraldehyde (Alfa) (2.21 g, 21.0 mmol) in CH₂Cl₂ (50 mL) was stirred at rt overnight. The reaction mixture was concentrated and the residue was dried in vacuo to give [3,3-dimethyl-but-(E)-ylideneamino]-acetic acid tert-butyl ester (4.29 g, 100%) as colorless oil which was used in the next step without further purification.

### Example 2

### Preparation of intermediate (Z)-2-(4-bromo-thiophen-3-yl)-3-(3-chloro-phenyl)-acrylonitrile

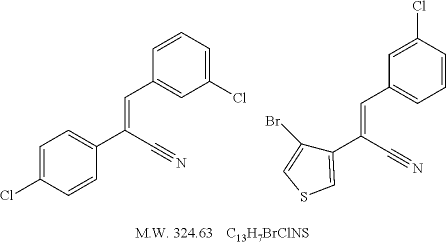

To a solution of 4-bromothiopene-2-acetonitrile (Matrix) (5 g, 24.7 mmol) and 3-chloro-benzaldehyde (Aldrich) (3.36 mL, 29.7 mmol) in methanol (200 mL) was slowly added a methanolic solution (Aldrich, 25 wt. %) of sodium methoxide (6.2 mL, 27.2 mmol). The reaction mixture was heated and stirred at room temperature for 1.5 h. The mixture became cloudy, and was filtered. The light yellow precipitate was washed with cold methanol, and then dried in vacu to give (Z)-2-(4-bromo-thiophen-3-yl)-3-(3-chloro-phenyl)-acrylonitrile as a light yellow solid (6.3 g, 79%).

### Example 3

### Preparation of intermediate rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid tert-butyl ester

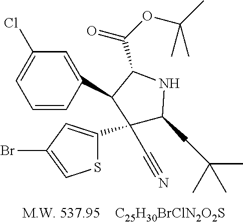

To a solution of [3,3-dimethyl-but-(E)-ylideneamino]-acetic acid tert-butyl ester (2.13 g, 10 mmol) and give (Z)-2-(4-bromo-thiophen-3-yl)-3-(3-chloro-phenyl)-acrylonitrile (2.59 g, 8 mmol) in dichloromethane (100 mL) were added triethyl amine (2.73 mL, 19.6 mmol) and AgF (1.51 g, 10 mmol). The mixture was stirred at room temperature for 48 h. The mixture was filtered through a short pad of silica gel. The silica gel was washed with ethyl acate. The filtrate was concentrated. The residue was purified by flash column chromatography (SiO₂, 1-20% of EtOAc in hexanes) to give rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid tert-butyl ester as a light yellow solid (2.6 g, 60%)

### Example 4

### Preparation of intermediate rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid trifluoroacetic acid

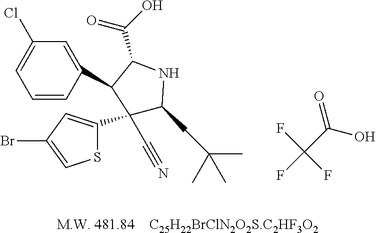

A solution of rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid tert-butyl ester (2.6 g, 4.8 mmol) in dichloromethane (7 mL) was added trifluoroacetic acid (3 mL). The reaction mixture was stirred at room temperature for 1 h, then concentrated. The residue was then triturated with ethyl ether hexanes, concentrated, dried under reduced pressure to give rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid trifluoroacetic acid as light yellow solid (2.5 g, 87%).

### Example 5

### Preparation of intermediate 2-((S)-2,2-dimethyl-[1,3]dioxolan-4-yl)-ethylamine

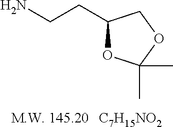

### Step A.

To a solution of (4S)-(+)-4-(2-hydroxyethyl)-2,2-dimethyl-1,3-dioxolane (Aldrich) (21.1 g, 0.14 mol) and triethylamine (40 mL, 0.28 mol) in dichloromethane (250 mL) at 0° C. was added methanesulfonyl chloride (13.4 mL, 0.17 mol) dropwise. The reaction mixture was stirred at 0° C. for 1.5 h, then water was added. The organic layer was separated, washed with water, brine, dried over MgSO₄, concentrated to give methanesulfonic acid 2-((S)-2,2-dimethyl-[1,3]dioxolan-4-yl)-ethyl ester as a yellow oil (31.7 g, 98%).

### Step B.

To a solution of methanesulfonic acid 2-((S)-2,2-dimethyl-[1,3]dioxolan-4-yl)-ethyl ester (31.7 g, 0.14 mol) in N,N-dimethylformamide (200 mL) was added NaN₃ (46 g, 0.71 mol). The reaction mixture was stirred at room temperature for 70 h. Then the mixture was partitioned between ethyl acetate and water. The organic layer was separated, washed with water, brine several times, dried over MgSO₄, concentrated to give (S)-4-(2-azido-ethyl)-2,2-dimethyl-[1,3]dioxolane as a yellow oil (21.3 g, 88%).

### Step C.

A suspension of (S)-4-(2-azido-ethyl)-2,2-dimethyl-[1,3]dioxolane as a yellow oil (18.7 g, 0.11 mol) and PtO₂ (2.5 g) in ethyl acetate (100 mL) was vigorously shaken in a Parr under atmosphere of H₂ (50 psi) for 18 h. The mixture was filtered through a short pad of celite. The filtrate was concentrated to give 2-((S)-2,2-dimethyl-[1,3]dioxolan-4-yl)-ethylamine as a colorless oil (14 g, 88%).

### Example 6

### Preparation of rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide

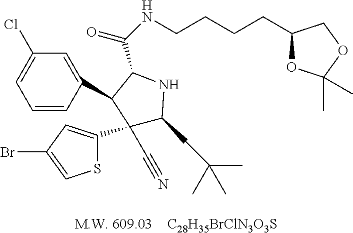

A mixture of rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid trifluoroacetic acid (0.3 g, 0.5 mmol) in dichloromethane (10 mL) was added 2-((S)-2,2-dimethyl-[1,3]dioxolan-4-yl)-ethylamine (0.22 g, 1.5 mmol), 2-(7-azabenzotriazol-1-yl)-N,N,N′,N′-tetramethyluronium hexafluorophosphate (HATU) (0.34 g, 0.9 mmol) and iPr₂NEt (0.44 mL, 2.5 mmol) respectively. The reaction mixture was stirred at room temperature for 18 h. The mixture was then diluted with CH₂Cl₂ and washed with water, brine. The organic phase was separated, filtered and dried over Na₂SO₄. The mixture was then concentrated and purified by SiO₂ flash column chromatography (20-100% of EtOAc in hexanes) to give rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide as a light yellow gum (0.26 g, 84%).

### Example 7

### Preparation of rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid ((S)-3,4-dihydroxy-butyl)-amide

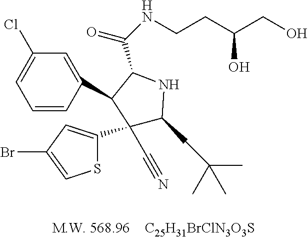

To a solution of rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide (0.26 g, 0.43 mol) in tetrahydrofuran (10 mL) was added aqueous HCl solution (1N, 1 mL). The reaction mixture was stirred at room temperature for 2 h, then concentrated. Then the residue was partitioned between ethyl acetate and water. The organic layer was separated, washed with water, aqueous saturated NaHCO₃, brine, dried over MgSO₄, concentrated, dried under reduced pressure to give rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid ((S)-3,4-dihydroxy-butyl)-amide as a light yellow solid (0.185 g, 75%).

HRMS (ES⁺) m/z Calcd for C₂₅H₃₁BrClN₃O₃S+H[(M+H)⁺]: 568.1031, found: 568.1032.

### Example 8

### Preparation of intermediate (Z)-2-(5-bromo-pyridin-2-yl)-3-(3-chloro-2-fluoro-phenyl)-acrylonitrile

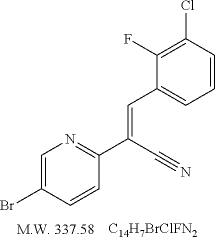

In a manner similar to the method described in Example 2, (5-bromopyridin-2-yl)acetonitrile(BetaPharma) (1 g, 5.1 mmol) was reacted with 3-chloro-2-fluorobenzaldehyde (0.96 g, 6.1 mmol), methanolic solution (25 wt %) of sodium methoxide (1.3 mL, 5.6 mmol) in methanol (30 mL) at 50° C. for 3 h to give (Z)-2-(5-bromo-pyridin-2-yl)-3-(3-chloro-2-fluoro-phenyl)-acrylonitrile as a grey solid (1.2 g, 71%).

### Example 9

### Preparation of intermediate rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid tert-butyl ester

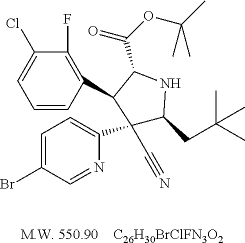

In a manner similar to the method described in Example 3, [3,3-dimethyl-but-(E)-ylideneamino]-acetic acid tert-butyl ester prepared in Example 1 (1.1 g, 5 mmol) was reacted with (Z)-2-(5-bromo-pyridin-2-yl)-3-(3-chloro-2-fluoro-phenyl)-acrylonitrile (1.2 g, 8.9 mmol) prepared in Example 8, AgF (0.54 g, 4.3 mmol), and triethylamine (1.24 mL, 29 mmol) in dichloromethane (80 mL) at room temperature for 18 h to give rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid tert-butyl ester as a light yellow solid (0.85 g, 44%).

### Example 10

### Preparation of intermediate rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid trifluoroacetic acid

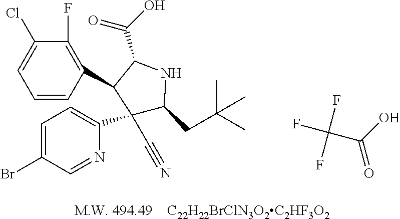

In a manner similar to the method described in Example 4, rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid tert-butyl ester prepared in Example 9 (0.85 g, 1.5 mmol) was reacted with trifluoroacetic acid in dichloromethane at room temperature to give rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid trifluoroacetic acid as a yellow solid (0.8 g, 85%).

### Example 11

### Preparation of rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide

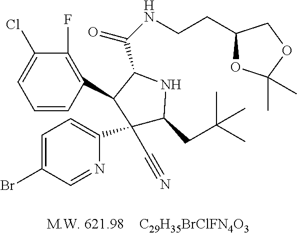

In a manner similar to the method described in Example 6, rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid trifluoroacetic acid prepared in Example 10 (0.35 g, 0.54 mmol) was reacted with 2-((S)-2,2-dimethyl-[1,3]dioxolan-4-yl)-ethylamine (0.24 g, 1.6 mmol), HATU (0.37 g, 0.98 mmol) and iPr₂NEt (0.47 mL, 2.7 mmol) in CH₂Cl₂ at room temperature to give rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide as a white foam (0.27 g, 75%).

### Example 12

### Preparation of rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide

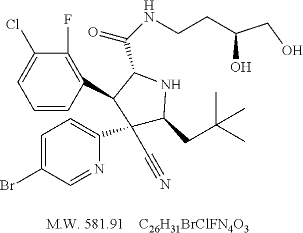

In a manner similar to the method described in Example 7, rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide prepared in Example 11 (0.25 g, 0.4 mmol) was reacted with aqueous HCl solution in tetrahydrofuran at room temperature to give rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide as a light yellow solid (0.21 g, 91%).

HRMS (ES⁺) m/z Calcd for C₂₆H₃₁BrClFN₄O₃+H [(M+H)⁺]: 581.1325, found: 581.1327.

### Example 13

### Preparation of intermediate (5-chloro-pyridin-2-yl)-acetonitrile

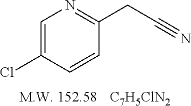

To the solution of 5-chloro-2-(chloromethyl)pyridine (CGenetech) (0.99 g, 6.1 mmol) in ethanol (8 mL) and H₂O (6 mL) was added KCN (1.03 g, 15.9 mmol). The reaction mixture was heated at 100° C. for 1 h. The mixture was cooled, and extracted with ethyl acetate. The organic layer was separated, washed with saturated aqueous NaHCO₃ solution, brine, dried over MgSO₄, and concentrated. The residue was purified by chromatography (EtOAc:hexanes=1:3) to give (5-chloro-pyridin-2-yl)acetonitrile as a yellow oil (0.64 g, 69%).

### Example 14

### Preparation of intermediate (Z)-3-(3-chloro-2-fluoro-phenyl)-2-(5-chloro-pyridin-2-yl)-acrylonitrile

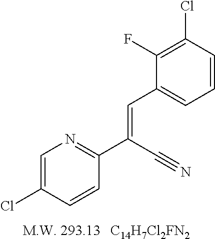

In a manner similar to the method described in Example 2, (5-chloropyridin-2-yl)acetonitrile (0.64 g, 4.2 mmol) was reacted with 3-chloro-2-fluorobenzaldehyde (0.8 g, 5.0 mmol), methanolic solution (25 wt %) of sodium methoxide (1.1 mL, 4.6 mmol) in methanol (30 mL) at 50° C. for 3 h to give (Z)-3-(3-chloro-2-fluoro-phenyl)-2-(5-chloro-pyridin-2-yl)-acrylonitrile as a light yellow solid (1.0 g, 81%).

### Example 15

### Preparation of intermediate rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid tert-butyl ester

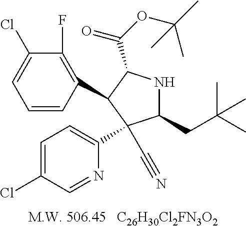

To a solution of [3,3-dimethyl-but-(E)-ylideneamino]-acetic acid tert-butyl ester prepared in Example 1 (0.95 g, 4.3 mmol) and (Z)-3-(3-chloro-2-fluoro-phenyl)-2-(5-chloro-pyridin-2-yl)-acrylonitrile (1.0 g, 3.4 mmol) prepared in Example 14 in dichloromethane (100 mL) were added triethylamine (1.19 mL, 8.5 mmol) and AgF (0.66 g, 5.2 mmol) sequentially.

The mixture was stirred at room temperature for 18 h. The mixture was then quenched with sat. NH₄Cl and extracted with CH₂Cl₂. The organic phase was separated, filtered through celite and dried over Na₂SO₄, and concentrated. The residue was dissolved into tert-butanol (20 mL), and DBU (3.3 mL, 24 mmol) was added. The mixture was heated at 100° C. for 18 h, then cooled to room temperature, and concentrated. The residue was partitioned between ethyl acetate and water. The organic layer were separated, dried over MgSO₄, and concentrated. The residue was purified by chromatography (EtOAc:hexanes=1;10, 1:5) to give rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid tert-butyl ester as a light yellow gum (1.3 g, 87%).

### Example 16

### Preparation of intermediate rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid trifluoroacetic acid

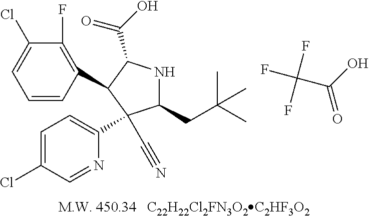

In a manner similar to the method described in Example 4, rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid tert-butyl ester prepared in Example 15 (1.3 g, 2.6 mmol) was reacted with trifluoroacetic acid in dichloromethane at room temperature to give rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid trifluoroacetic acid as a light yellow solid (1.15 g, 79%).

### Example 17

### Preparation of rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid [2-((S)-2,2-dimethyl-[1,3]dioxolan-4-yl)-ethyl]-amide

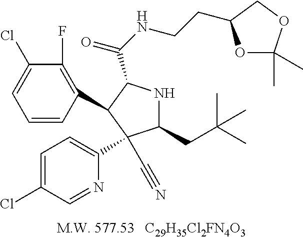

In a manner similar to the method described in Example 6, rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid trifluoroacetic acid prepared in Example 16 (0.45 g, 0.80 mmol) was reacted with 2-((S)-2,2-dimethyl-[1,3]dioxolan-4-yl)-ethylamine (0.35 g, 2.4 mmol), HATU (0.54 g, 1.4 mmol) and iPr₂NEt (0.69 mL, 4.0 mmol) in CH₂Cl₂ at room temperature to give rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid [2-((S)-2,2-dimethyl-[1,3]dioxolan-4-yl)-ethyl]-amide as a light yellow gum (0.3 g, 65%).

### Example 18

### Preparation of rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide

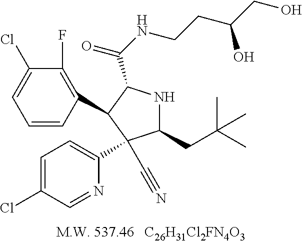

In a manner similar to the method described in Example 7, rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide prepared in Example 17 (0.3 g, 0.52 mmol) was reacted with aqueous HCl solution in tetrahydrofuran at room temperature to give rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide as a light yellow solid (0.25 g, 89%).

HRMS (ES⁺) m/z Calcd for C₂₆H₃₁Cl₂FN₄O₃+H[(M+H)⁺]: 537.1830, found: 537.1828.

### Example 19

### Preparation of chiral-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide

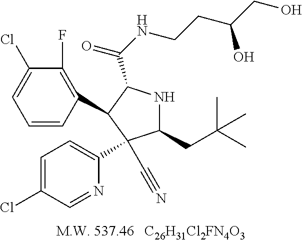

Rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide (0.22 g) was separated by chiral SFC chromatography to provide chiral-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide as a light yellow solid (63 mg, 29%) and chiral-(2S,3R,4R,5R)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide as a light yellow solid (62 mg, 28%).

HRMS (ES⁺) m/z Calcd for C₂₆H₃₁Cl₂FN₄O₃+H[(M+H)⁺]: 537.1830, found: 537.1830.

### Example 20

### Preparation of intermediate (5-chloro-3-fluoro-pyridin-2-yl)-acetonitrile

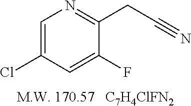

### Step A

To a solution of ethyl cyanoacetate (Aldrich) (4 g, 35.4 mmol) in anhydrous DMSO (30 mL) at 0° C. was added slowly NaH (60%, 1.42 g, 35.6 mmol). The mixture was stirred at 0° C. for 0.5 h, then 5-chloro-2,3-difluoropyridine (Combi-Blocks) (5.3 g, 35.6 mmol) was added. The reaction mixture was stirred at room temperature for 18 h. Water was added. The organic layer was separated, the aqueous layer was then extracted with ethyl acetate twice. The combined organic layers were washed with brine, dried over MgSO₄, concentrated. The residue was purified by chromatography (EtOAc:hexanes=1:4, 1:1) to give (5-chloro-3-fluoro-pyridin-2-yl)-acetonitrile as a yellow gum (3.2 g, 37%).

### Step B

To the solution of (5-chloro-3-fluoro-pyridin-2-yl)-acetonitrile (2.8 g, 11.5 mmol) in DMSO (30 mL) was added NaCl (2 g, 34 mmol). The reaction mixture was heated at 170° C. for 2 h. The mixture was partitioned between ethyl acetate and water. Organic layer was separated, the aqueous layer was extracted with ethyl acetate. The combined organic layers were washed with water, brine, dried over MgSO₄, and concentrated. The residue was purified by chromatography (EtOAc:hexanes=1:4, 1:3) to give (5-chloro-3-fluoro-pyridin-2-yl)-acetonitrile as a brown oil (0.85 g, 43%).

### Example 21

### Preparation of intermediate (Z)-3-(3-chloro-2-fluoro-phenyl)-2-(5-chloro-3-fluoro-pyridin-2-yl)-acrylonitrile

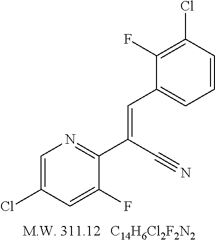

In a manner similar to the method described in Example 2, (5-chloro-3-fluoro-pyridin-2-yl)-acetonitrile (0.85 g, 5.0 mmol) was reacted with 3-chloro-2-fluorobenzaldehyde (0.8 g, 5.0 mmol), methanolic solution (25 wt %) of sodium methoxide (1.1 g, 5 mmol) in methanol (50 mL) at room temperature for 3 h to give (Z)-3-(3-chloro-2-fluoro-phenyl)-2-(5-chloro-3-fluoro-pyridin-2-yl)-acrylonitrile as a white solid (1.24 g, 80%).

### Example 22

### Preparation of intermediate rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid tert-butyl ester

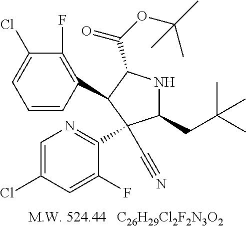

To a solution of [3,3-dimethyl-but-(E)-ylideneamino]-acetic acid tert-butyl ester prepared in Example 1 (1.1 g, 5 mmol) and (Z)-3-(3-chloro-2-fluoro-phenyl)-2-(5-chloro-3-fluoro-pyridin-2-yl)-acrylonitrile (1.24 g, 4 mmol) prepared in Example 21 in dichloromethane (100 mL) were added triethylamine (1.39 mL, 10 mmol) and AgF (0.77 g, 6.1 mmol) sequentially. The mixture was stirred at room temperature for 18 h. The mixture was then quenched with sat. NH₄Cl and extracted with CH₂Cl₂. The organic phase was separated, filtered through celite and dried over Na₂SO₄, and concentrated. The residue was dissolved into tert-butanol (10 mL), and DBU (4.8 mL, 32 mmol) was added. The mixture was heated at 100° C. for 18 h, then cooled to room temperature, and concentrated. The residue was partitioned between ethyl acetate and water. The organic layer were separated, dried over MgSO₄, and concentrated. The residue was purified by chromatography (EtOAc:hexanes=1;10, 1:5) to give rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid tert-butyl ester as a light yellow gum (1.0 g, 48%).

### Example 23

### Preparation of intermediate rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid trifluoroacetic acid

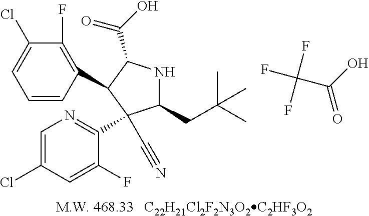

In a manner similar to the method described in Example 4, rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid tert-butyl ester prepared in Example 22 (1.0 g, 1.9 mmol) was reacted with trifluoroacetic acid in dichloromethane at room temperature to give rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid trifluoroacetic acid as a yellow solid (1.0 g, 90%).

### Example 24

### Preparation of rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid [2-((S)-2,2-dimethyl-[1,3]dioxolan-4-yl)-ethyl]-amide

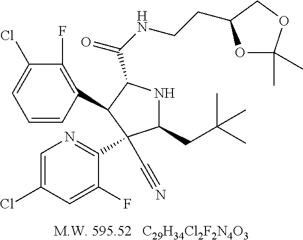

In a manner similar to the method described in Example 6, rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid trifluoroacetic acid prepared in Example 23 (0.45 g, 0.77 mmol) was reacted with 24(S)-2,2-dimethyl-[1,3]dioxolan-4-yl)-ethylamine (0.34 g, 2.3 mmol), HATU (0.53 g, 1.4 mmol) and iPr₂NEt (0.67 mL, 3.9 mmol) in CH₂Cl₂ at room temperature to give rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid [2-((S)-2,2-dimethyl-[1,3]dioxolan-4-yl)-ethyl]-amide as a light yellow gum (0.2 g, 44%).

### Example 25

### Preparation of rac-(2R,3S,4S,5S)-3-(3-chloro-2-fluoro-phenyl)-4-(5-chloro-3-yl)-4-cyano-5-(2,2-dimethyl-propyl)-pyrrolidine-2-carboxylic acid ((S)-3,4-dihydroxy-butyl)-amide

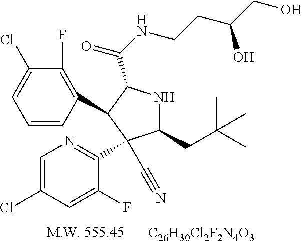

In a manner similar to the method described in Example 7, rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid ((S)-3,4-dihydroxy-butyl)-amide prepared in Example 24 (0.2 g, 0.33 mmol) was reacted with aqueous HCl solution in tetrahydrofuran at room temperature to give rac-(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carboxylic acid ((S)-3,4-dihydroxy-butyl)-amide as a light yellow solid (0.1 g, 53%).

HRMS (ES⁺) m/z Calcd for C₂₆H₃₀Cl₂F₂N₄O₃+H[(M+H)⁺]: 555.1736, found: 555.1736.

### Example 26

### Preparation of intermediate (5-bromo-pyrimidin-2-yl)-acetonitrile

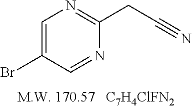

### Step A

To a solution of tert-butyl cyanoacetate (Alfa) (1.5 g, 11 mmol) in anhydrous tetrahydrofuran (100 mL) at 0° C. was added slowly NaH (60%, 1.0 g, 25 mmol). The mixture was stirred at 0° C. for 0.5 h, then 5-bromo-2-chloropyrimidine (TCI-US) (2.5 g, 13 mmol) was added. The reaction mixture was stirred at room temperature for 18 h. Water was added. The mixture was partitioned between ethyl acetate and water. The organic layer was separated, the aqueous layer was then extracted with ethyl acetate. The combined organic layers were washed with brine, dried over MgSO₄, concentrated. The residue was trituated with ethyl acetate and hexanes, and the mixture was filtered to give (5-bromo-pyrimidin-2-yl)-cyano-acetic acid tert-butyl ester as a yellow precipitate (3.2 g, 98%).

### Step B

To the solution of (5-bromo-pyrimidin-2-yl)-cyano-acetic acid tert-butyl ester (3.2 g, 10 mmol) in dichloromethane (30 mL) was added trifluoroacetic acid (10 mL). The reaction mixture was stirred at room temperature for 4 h. The mixture was concentrated. To the residue was added water, and the “pH” of the mixture was adjusted to neutral by saturated aqueous NaHCO₃ solution. The mixture was then extracted with ethyl acetate. Organic layer was separated, the aqueous layer was extracted with ethyl acetate. The combined organic layers were washed with water, brine, dried over MgSO₄, and concentrated. The residue was purified by chromatography (EtOAc:hexanes=1:3, 1:2, 1;1) to give (5-bromo-pyrimidin-2-yl)-acetonitrile as a white solid (1.2 g, 71%).

### Example 27

### Preparation of intermediate (Z)-2-(5-bromo-pyrimidin-2-yl)-3-(3-chloro-2-fluoro-phenyl)-acrylonitrile

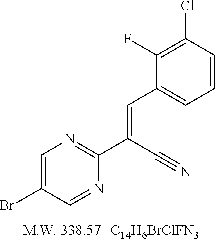

In a manner similar to the method described in Example 2, (5-bromo-pyrimidin-2-yl)-acetonitrile (2.2 g, 13 mmol) was reacted with 3-chloro-2-fluorobenzaldehyde (2.2 g, 14 mmol), methanolic solution (25 wt %) of sodium methoxide (3 mL, 13 mmol) in methanol (30 mL) at room temperature for 3 h to give (Z)-2-(5-bromo-pyrimidin-2-yl)-3-(3-chloro-2-fluoro-phenyl)-acrylonitrile as a white solid (2.9 g, 66%).

### Example 28

### Preparation of intermediate rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid tert-butyl ester

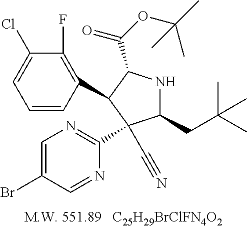

To a solution of [3,3-dimethyl-but-(E)-ylideneamino]-acetic acid tert-butyl ester prepared in Example 1 (1.1 g, 5 mmol) and (Z)-2-(5-bromo-pyrimidin-2-yl)-3-(3-chloro-2-fluoro-phenyl)-acrylonitrile (1.33 g, 4 mmol) prepared in Example 27 in dichloromethane (100 mL) were added triethylamine (1.39 mL, 10 mmol) and AgF (0.77 g, 6.1 mmol) sequentially. The mixture was stirred at room temperature for 18 h. The mixture was then quenched with sat. NH₄Cl and extracted with CH₂Cl₂. The organic phase was separated, filtered through celite and dried over Na₂SO₄, and concentrated. The residue was dissolved into tert-butanol (10 mL), and DBU (4.8 mL, 32 mmol) was added. The mixture was heated at 100° C. for 18 h, then cooled to room temperature, and concentrated. The residue was partitioned between ethyl acetate and water. The organic layer were separated, dried over MgSO₄, and concentrated. The residue was purified by chromatography (EtOAc:hexanes=1:3) to give rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid tert-butyl ester as a yellow gum (0.45 g, 20%).

### Example 29

### Preparation of intermediate rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid trifluoroacetic acid

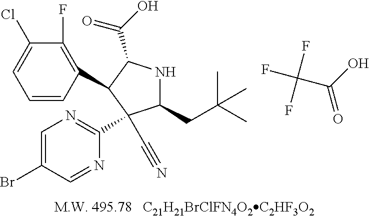

In a manner similar to the method described in Example 4, rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid tert-butyl ester prepared in Example 28 (0.45 g, 0.82 mmol) was reacted with trifluoroacetic acid in dichloromethane at room temperature to give rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid trifluoroacetic acid as a light yellow solid (0.36 g, 88%).

### Example 30

### Preparation of rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide

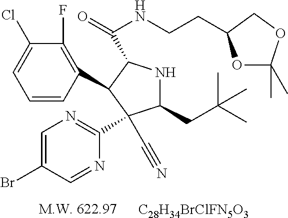

In a manner similar to the method described in Example 6, rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid trifluoroacetic acid prepared in Example 29 (0.36 g, 0.71 mmol) was reacted with 2-((S)-2,2-dimethyl-[1,3]dioxolan-4-yl)-ethylamine (0.31 g, 2.1 mmol), HATU (0.48 g, 1.28 mmol) and iPr₂NEt (0.62 mL, 3.55 mmol) in CH₂Cl₂ at room temperature to give rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid [2-((S)-2,2-dimethyl-[1,3]dioxolan-4-yl)-ethyl]-amide as a white gum (0.28 g, 64%).

### Example 31

### Preparation of rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide

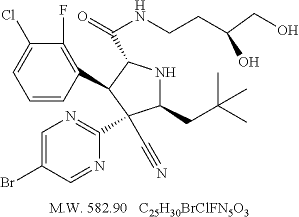

In a manner similar to the method described in Example 7, rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide prepared in Example 30 (0.28 g, 0.45 mmol) was reacted with aqueous HCl solution in tetrahydrofuran at room temperature to give rac-(2R,3S,4S,5S)-4-(5-bromo-pyrimidin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide as a light yellow solid (0.21 g, 81%).

HRMS (ES⁺) m/z Calcd for C₂₅H₃₀BrClFN₅O₃+H[(M+H)⁺]: 582.1278, found: 582.1282.

### Example 32

### Preparation of rac-4-{[(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carbonyl]amino}-benzoic acid methyl ester

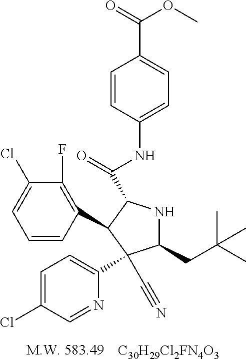

In a manner similar to the method described in Examples 6, rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid trifluoroacetic acid prepared in Example 16 (0.3 g, 0.52 mmol) was reacted with methyl 4-aminobenzoate (Acros)(0.12 g, 0.79 mmol), HATU (0.36 g, 0.96 mmol) and iPr₂NEt (0.23 mL, 1.3 mmol) in CH₂Cl₂ at room temperature to give rac-4-{[(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carbonyl]-amino}-benzoic acid methyl ester as a white solid (50 mg, 16%).

HRMS (ES⁺) m/z Calcd for C₃₀H₂₉Cl₂FN₄O₃+H[(M+H)⁺]: 583.1674, found: 583.1674.

### Example 33

### Preparation of rac-4-{[(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carbonyl]-amino}-benzoic acid

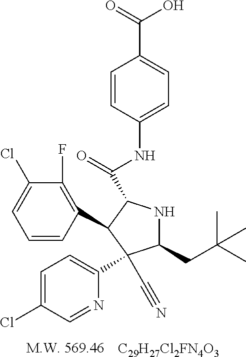

To a solution of rac-4-{[(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carbonyl]-amino}-benzoic acid methyl ester prepared in Example 32 (30 mg, 0.05 mmol) in tetrahydrofuran (3 mL) was added an aqueous solution (1 N) of NaOH (3 mL, 3 mmol) and methanol (1 mL). The reaction mixture was stirred at room temperature for 18 h, and the “pH” of the solution was adjusted to 5-6 by aqueous HCl solution. The mixture was extracted ethyl acetate twice. The combined organic extracts were washed with water, brine, dried over MgSO₄, and concentrated. The residue was triturate with dichlormethane and hexanes to give rac-4-{[(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carbonyl]-amino}-benzoic acid as a off white solid (28 mg, 82%).

HRMS (ES⁺) m/z Calcd for C₂₉H₂₇Cl₂FN₄O₃+H[(M+H)⁺]: 569.1517, found: 569.1518.

### Example 34

### Preparation of rac-4-[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]amino}-benzoic acid methyl ester

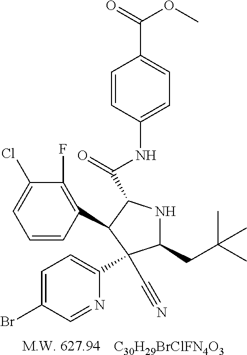

In a manner similar to the method described in Examples 6, rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid trifluoroacetic acid prepared in Example 10 (0.24 g, 0.39 mmol) was reacted with methyl 4-aminobenzoate (Acros)(0.59 g, 3.9 mmol), HATU (0.27 g, 0.7 mmol) and iPr₂NEt (0.17 mL, 0.98 mmol) in CH₂Cl₂ at room temperature to give rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid methyl ester as a white solid (50 mg, 20%).

HRMS (ES⁺) m/z Calcd for C₃₀H₂₉BrClFN₄O₃+H[(M+H)⁺]: 627.1169, found: 627.1167.

### Example 35

### Preparation of rac-4-[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid

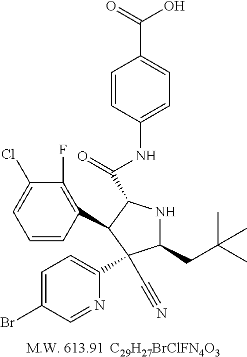

To a solution rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid methyl ester prepared in Example 34 (35 mg, 0.056 mmol) in tetrahydrofuran (3 mL) was added an aqueous solution (1 N) of NaOH (3 mL, 3 mmol) and methanol (1 mL). The reaction mixture was stirred at room temperature for 18 h, and the “pH” of the solution was adjusted to 5-6 by aqueous HCl solution. The mixture was extracted ethyl acetate twice. The combined organic extracts were washed with water, brine, dried over MgSO₄, and concentrated to rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid as a white solid (28 mg, 82%).

HRMS (ES⁺) m/z Calcd for C₂₉H₂₇BrClFN₄O₃+H[(M+H)⁺]: 627.1169, found: 627.1167.

### Example 36

### Preparation of intermediate (Z)-3-(3-chloro-phenyl)-2-pyridin-3-yl-acrylonitrile

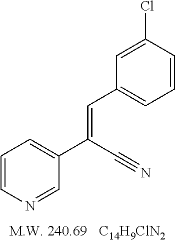

To a solution of 3-chlorobenzaldehyde (0.7 g, 5 mmol) and 3-pyridylacetonitrile (TCI-Japan) (0.59 g, 5 mmol) in iPrOH (20 mL) was added aqueous solution (2 N) of NaOH (0.3 mL, 0.6 mmol). The reaction mixture was stirred at room temperature for 48 h. The mixture was partitioned between ethyl acetate and water. The organic layer was separated, washed with water, brine, dried over Na₂SO₄, and concentrated. The residue was purified by flash column chromatography (10-70% of AcOEt in hexanes) to give (Z)-3-(3-chloro-phenyl)-2-pyridin-3-yl-acrylonitrile as a yellow foam (0.33 g, 28%).

### Example 37

### Preparation of intermediate rac-(2R,3R,4R,5S)-3-(3-chloro-phenyl)-4-cyano-5-(2,2-dimethyl-propyl)-4-pyridin-3-yl-pyrrolidine-2-carboxylic acid tert-butyl ester

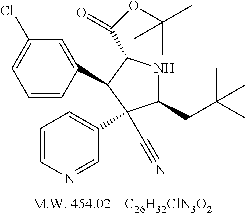

In a manner similar to the method described in Example 3, [3,3-dimethyl-but-(E)-ylideneamino]-acetic acid tert-butyl ester prepared in Example 1 (0.42 g, 2 mmol) was reacted with (Z)-3-(3-chloro-phenyl)-2-pyridin-3-yl-acrylonitrile (0.31 g, 1.3 mmol) prepared in Example 36, AgF (0.25 g, 2 mmol), and triethylamine (0.4 g, 4 mmol) in 1,2-dichloroethane (10 mL) at room temperature for 18 h to give rac-(2R,3R,4R,5S)-3-(3-chloro-phenyl)-4-cyano-5-(2,2-dimethyl-propyl)-4-pyridin-3-yl-pyrrolidine-2-carboxylic acid tert-butyl ester as a light yellow solid (0.29 g, 49%).

HRMS(ES⁺) m/z Calcd for C₂₆H₃₂ClN₃O₂+H[(M+H): 454.2256; Found: 454.2258.

### Example 38

### Preparation of intermediate rac-(2R,3R,4R,5S)-3-(3-chloro-phenyl)-4-cyano-5-(2,2-dimethyl-propyl)-4-pyridin-3-yl-pyrrolidine-2-carboxylic acid

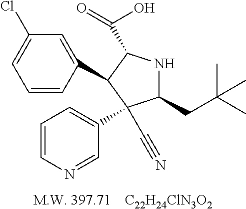

A solution of rac-(2R,3R,4R,5S)-3-(3-chloro-phenyl)-4-cyano-5-(2,2-dimethyl-propyl)-4-pyridin-3-yl-pyrrolidine-2-carboxylic acid tert-butyl ester (0.25 g, 0.55 mmol) in conc. H₂SO₄ (2 mL) was stirred at room temperature for 2 h. The mixture was then poured into ice and extracted with ethyl acetate. The organic layer was separated, dried over Na₂SO₄, and concentrated. The residue was purified by chromatography (15-25% EtOAc in hexanes) to give rac-(2R,3R,4R,5S)-3-(3-chloro-phenyl)-4-cyano-5-(2,2-dimethyl-propyl)-4-pyridin-3-yl-pyrrolidine-2-carboxylic acid as a white solid (0.19 g, 87%).

HRMS(ES⁺) m/z Calcd for C₂₂H₂₄ClN₃O₂+H[(M−H):398.1628; Found: 398.1630.

### Example 39

### Preparation of rac-(2R,3R,4R,5S)-3-(3-chloro-phenyl)-4-cyano-5-(2,2-dimethyl-propyl)-4-pyridin-3-yl-pyrrolidine-2-carboxylic acid ((S)-3,4-dihydroxy-butyl)-amide

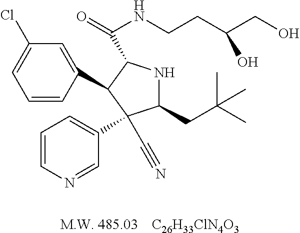

A mixture of rac-(2R,3R,4R,5S)-3-(3-chloro-phenyl)-4-cyano-5-(2,2-dimethyl-propyl)-4-pyridin-3-yl-pyrrolidine-2-carboxylic acid (80 mg, 0.2 mmol), 2-((S)-2,2-dimethyl-[1,3]dioxolan-4-yl)-ethylamine (44 mg, 0.3 mmol), HATU (114 mg, 0.3 mmol) and iPr₂NEt (0.1 mL, 1 mmol) in CH₂Cl₂ (2 mL) was stirred at room temperature for 1 h. The mixture was then diluted with CH₂Cl₂ and washed with water, brine. The organic phase was separated, filtered and dried over Na₂SO₄. The mixture was then concentrated and to the residue was added PPTS (cat) and methanol (2 mL). The reaction mixture was heated under microwave irradiation in CEM microwave reactor at 120° C. for 5 min. The mixture was concentrated and the residue was diluted with EtOAc and washed with water, brine. The organic phase was separated, dried over Na₂SO₄, and concentrated. The residue was purified by SiO₂ flash column chromatography (5% of MeOH in EtOAc) to give rac-(2R,3R,4R,5S)-3-(3-chloro-phenyl)-4-cyano-5-(2,2-dimethyl-propyl)-4-pyridin-3-yl-pyrrolidine-2-carboxylic acid ((S)-3,4-dihydroxy-butyl)-amide as a white amorphous (63 mg, 61%).

HRMS (ES⁺) m/z Calcd for C₂₆H₃₃ClN₄O₃+H[(M+H)⁺]: 485.2314; Found: 485.2311.

### Example 40

### Preparation of intermediate (Z)-3-(3-chloro-phenyl)-2-(6-chloro-pyridin-3-yl)-acrylonitrile

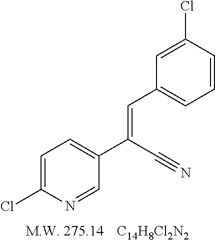

To a solution of 3-chlorobenzaldehyde (1.4 g, 10 mmol) and 2-chloro-5-(cyanomethyl)pyridine (Matrix)(1.52 g, 10 mmol) in iPrOH (20 mL) was added aqueous solution (2 N) of NaOH (0.6 mL, 1.2 mmol). The reaction mixture was stirred at room temperature for 20 h. The mixture was partitioned between ethyl acetate and water. The organic layer was separated, washed with water, brine, dried over Na₂SO₄, and concentrated. The residue was purified by flash column chromatography (10-70% of AcOEt in hexanes) to give (Z)-3-(3-chloro-phenyl)-2-(6-chloro-pyridin-3-yl)-acrylonitrile as a white solid (1.85 g, 67%).

HRMS (ES⁺) m/z Calcd for C₁₄H₈Cl₂N₂+H[(M+H): 275.0138; Found: 275.0137.

### Example 41

### Preparation of intermediate rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid tert-butyl ester

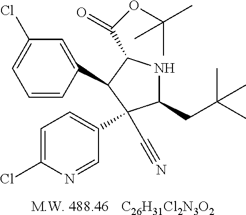

In a manner similar to the method described in Example 3, [3,3-dimethyl-but-(E)-ylideneamino]-acetic acid tert-butyl ester prepared in Example 1 (0.53 g, 2.5 mmol) was reacted with (Z)-3-(3-chloro-phenyl)-2-(6-chloro-pyridin-3-yl)-acrylonitrile (0.55 g, 2 mmol) prepared in Example 40, AgF (0.32 g, 2.5 mmol), and triethylamine (0.5 g, 5 mmol) in 1,2-dichloroethane (20 mL) at room temperature for 18 h to give rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid tert-butyl ester as a light yellow solid (0.13 g, 14%).

HRMS(ES⁺) m/z Calcd for C₂₆H₃₁Cl₂N₃O₂+H[(M+H): 488.1866; Found: 488.1864.

### Example 42

### Preparation of intermediate rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid

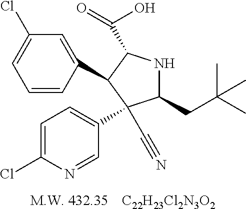

A solution of rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid tert-butyl ester (0.36 g, 0.73 mmol) in conc. H₂SO₄ (1 mL) and acetonitrile (3 mL) was heated under microwave irradiaition at 120° C. for 10 min. The mixture was then poured into ice-water, and the “pH” was adjusted to neutral by aqueous NaOH solution. The mixture was extracted with ethyl acetate. The organic layer was separated, dried over Na₂SO₄, and concentrated. The residue was purified by chromatography (15-25% EtOAc in hexanes) to give rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid as a white amorphous (0.21 g, 24%).

HRMS(ES⁺) m/z Calcd for C₂₂H₂₃Cl₂N₃O₂+H[(M+H): 432.1240; Found: 432.1241.

### Example 43

### Preparation of rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid [2-((S)-2,2-dimethyl-[1,3]dioxolan-4-yl)-ethyl]-amide

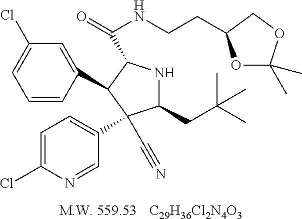

In a manner similar to the method described in Example 6, rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid (0.2 g, 0.46 mmol) was reacted with 2-((S)-2,2-dimethyl-[1,3]dioxolan-4-yl)-ethylamine (0.1 g, 0.69 mmol), HATU (0.26 g, 0.69 mmol) and iPr₂NEt (0.12 mL, 0.92 mmol) in CH₂Cl₂ at room temperature to give rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid [2-((S)-2,2-dimethyl-[1,3]dioxolan-4-yl)-ethyl]-amide as a white amorphous (0.21 g, 81%).

HRMS(ES⁺) m/z Calcd for C₂₉H₃₆Cl₂N₄O₃+H[(M+H): 559.2237; Found: 559.2234.

### Example 44

### Preparation of rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid ((S)-3,4-dihydroxy-butyl)-amide

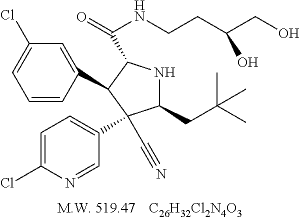

To a solution of rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid [2-((S)-2,2-dimethyl-[1,3]dioxolan-4-yl)-ethyl]-amide (70 mg, 0.27 mmol) in methanol (2 mL) was added PPTS (cat.). The reaction mixture was heated under microwave irradiation in CEM microwave reactor at 120° C. for 5 min. The mixture was concentrated and the residue was diluted with EtOAc and washed with water, brine. The organic phase was separated, dried over Na₂SO₄, and concentrated. The residue was purified by SiO₂ flash column chromatography (50-100% EtOAc in hexanes) to give rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid ((S)-3,4-dihydroxy-butyl)-amide as a white amorphous (47 mg, 73%).

HRMS(ES⁺) m/z Calcd C₂₆H₃₂Cl₂N₄O₃+H[(M+H): 519.1924; Found: 519.1925.

### Example 45

### Preparation of rac-4-{[(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carbonyl]-amino}-3-methoxy-benzoic acid methyl ester

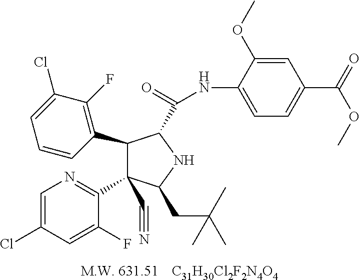

To a stirred solution of (2R,3S,4S,5S)-3-(3-chloro-2-fluorophenyl)-4-(5-chloro-3-fluoropyridin-2-yl)-4-cyano-5-neopentylpyrrolidine-2-carboxylic acid (214 mg, 0.457 mmol) in 4 ml methylene chloride, diphenylphosphinic chloride (Aldrich, 216, 0.174 ml) was added followed by DIPEA (Aldrich, 177 mg, 0.239 ml). The solution was stirred for 10 min. at rt and then methyl 4-amino-3-methoxybenzoate (Aldrich, 91 mg, 0.50 mmol) was added and the mixture was stirred at rt overnight. The solvent was reduced to about 3 ml and loaded on a 40 g silica gel column and eluted on a esco machine (3-5% EtOAc/CH2Cl2) to givea solid, which was again chromatographied on a reverse phase column (50-95% CH3CN/H2O) to give 55 mg white solid.

MS (ES⁺) m/z Calcd for C₃₁H₃₀Cl₂F₂N₄O₄+H[(M+H)⁺]: 631, found: 631.

### Example 46

### Preparation of rac-4-{[(2R,3S,4S,5S)-3-(3-Chloro-2-fluoro-phenyl)-4-(5-chloro-3-fluoro-pyridin-2-yl)-4-cyano-5-(2,2-dimethyl-propyl)-pyrrolidine-2-carbonyl]-amino}-3-fluoro-benzoic acid methyl ester

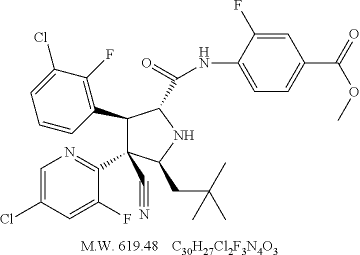

In a 25 mL round-bottomed flask, (2R,3S,4S,5S)-3-(3-chloro-2-fluorophenyl)-4-(5-chloro-3-fluoropyridin-2-yl)-4-cyano-5-neopentylpyrrolidine-2-carboxylic acid 2,2,2-trifluoroacetic acid (1:1) salt(150 mg, 258 μmol, Eq: 1.00), was combined with CH₂Cl₂ (3 ml) to give a suspension. N-ethyl-N-isopropylpropan-2-amine (117 mg, 157 μL, 902 mol, Eq: 3.5) and diphenylphosphinic chloride (Aldrich, 152 mg, 123 μL, 644 μmol, Eq: 2.5) were added and the reaction was stirred at RT for 10 minutes. Methyl 4-amino-3-fluorobenzoate (Aldrich, 43.6 mg, 258 μmol, Eq: 1.00) was added and the reaction mixture was stirred at RT for 4 days and then concentrated on Rotor Vac.

The crude material was dissolved in DMSO and was purified by preparative HPLC (65-100% ACN/H2O, 0.1% TFA). The fractions was combined, concentrated and freeze dried to give a yellow solid (7.5 mg, 4.7% yield) as desired product. MS (ES⁺) m/z Calcd for C₃₀H₂₇Cl₂F₃N₄O₃+H[(M+H)⁺]: 619, found: 619.

### Example 47

### In Vitro Activity Assay

The ability of the compounds to inhibit the interaction between p53 and MDM2 proteins was measured by an HTRF (homogeneous time-resolved fluorescence) assay in which recombinant GST-tagged MDM2 binds to a peptide that resembles the MDM2-interacting region of p53 (Lane et al.). Binding of GST-MDM2 protein and p53-peptide (biotinylated on its N-terminal end) is registered by the FRET (fluorescence resonance energy transfer) between Europium (Eu)-labeled anti-GST antibody and streptavidin-conjugated Allophycocyanin (APC).

Test is performed in black flat-bottom 384-well plates (Costar) in a total volume of 40 uL containing:90 nM biotinylate peptide, 160 ng/ml GST-MDM2, 20 nM streptavidin-APC (PerkinElmerWallac), 2 nM Eu-labeled anti-GST-antibody (PerkinElmerWallac), 0.2% bovine serum albumin (BSA), 1 mM dithiothreitol (DTT) and 20 mM Tris-borate saline (TBS) buffer as follows: Add 10 uL of GST-MDM2 (640 ng/ml working solution) in reaction buffer to each well. Add 10 uL diluted compounds (1:5 dilution in reaction buffer) to each well, mix by shaking. Add 20 uL biotinylated p53 peptide (180 nM working solution) in reaction buffer to each well and mix on shaker. Incubate at 37° C. for 1 h. Add 20 uL streptavidin-APC and Eu-anti-GST antibody mixture (6 nM Eu-anti-GST and 60 nM streptavidin-APC working solution) in TBS buffer with 0.2% BSA, shake at room temperature for 30 minutes and read using a TRF-capable plate reader at 665 and 615 nm (Victor 5, Perkin ElmerWallac). If not specified, the reagents were purchased from Sigma Chemical Co.

Activity data for some of the Example compounds expressed as IC₅₀ :bsa:0.02% are as follows:

| Example Number | IC₅₀: bsa: 0.02% |
|---|---|
| 7 | 5.864 |
| 25 | 1.232 |
| 34 | 0.201 |
| 39 | 1.968 |
| 44 | 0.061 |

What is claimed:

1. A compound of the formula 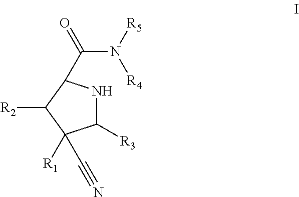
wherein
R₁ is a substituted or unsubstituted heteroaryl selected from 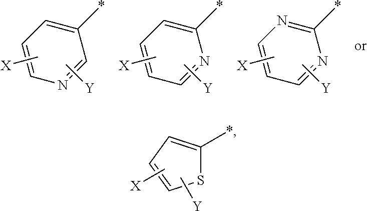
X is selected from the group consisting of H, F, Cl, Br and I,
Y is H or F,
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄ and R₅ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—(CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂)ₘ—(CH₂)ₙ—SO₂NR′R″, R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle, or substituted hetereocycle.
and in the case of R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle,
m, n and p are independently 0 to 6
and the pharmaceutically acceptable salts and esters thereof.

2. The compound of claim 1 wherein R₂ is selected from 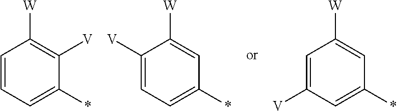
wherein
W is F, Cl or Br,
V is H or F,
and R₃ is a substituted lower alkyl selected from 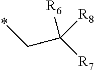
where R₆, R₇ are both methyl, or linked to form a cyclopropyl, cyclobutyl, cyclopentyl or acyclohexyl group,
R₈ is (CH₂)_{q}—R_{9,}
q is 0, 1 or 2 and
R₉ is selected from hydrogen, hydroxyl, lower alkyl, lower alkoxy, aryl, substituted aryl. hetereoaryl, substituted heteroaryl, hetereocycle or substituted heterocycle.

3. A compound of the formula 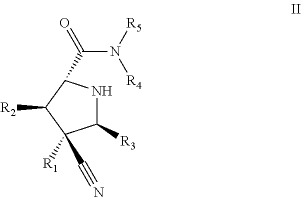
wherein
R₁ is a substituted or unsubstituted heteroaryl selected from 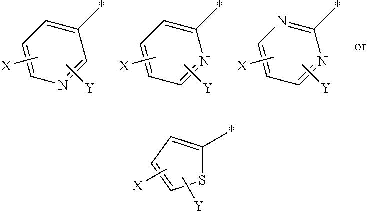
X is selected from the group consisting of H, F, Cl, Br and I
Y is H or F
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄ and R₅ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O()ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle, or substituted hetereocycle,
and in the case of R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle,
m, n and p are independently 0 to 6
and the pharmaceutically acceptable salts and esters thereof.

4. The compound of claim 3 wherein R₂ is selected from 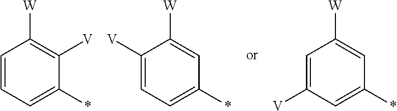
wherein
W is F, Cl or Br,
V is H or F,
and R₃ is a substituted lower alkyl selected from 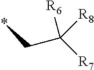
where R₆, R₇ are both methyl, or linked to form a cyclopropyl, cyclobutyl, cyclopentyl or acyclohexyl group,
R₈ is (CH₂)_{q}—R₉,
q is 0, 1 or 2,
and
R₉ is selected from hydrogen, hydroxyl, lower alkyl, lower alkoxy, aryl, substituted aryl. hetereoaryl, substituted heteroaryl, hetereocycle or substituted heterocycle.

5. The compound of claim 3 wherein
R₁ is a substituted or unsubstituted heteroaryl selected from 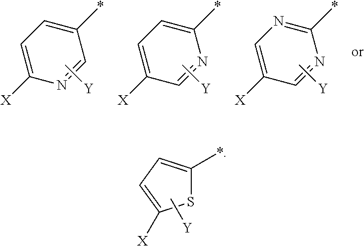
X is selected from the group consisting of F, Cl, Br,
Y is H or F,
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄ is hydrogen and R₅ is (CH₂)ₙ—R′,
n is 0 or 1 and
R′ is selected from aryl, substituted aryl, hetereoaryl, substituted heteroaryl, hetereocycle or substituted heterocycle
and the pharmaceutically acceptable salts and esters thereof.

6. The compound of claim 5 wherein R₂ is a substituted aryl selected from 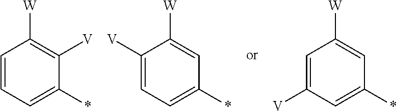
wherein W is F, Cl or Br and
V is H or F.

7. The compound of claim 6 wherein R₃ is a substituted lower alkyl selected from 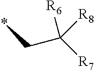
where R₆, R₇ are both methyl, or linked to form a cyclopropyl, cyclobutyl, cyclopentyl or acyclohexyl group,
R₈ is (CH₂)_{q}—R₉
q is 0, 1 or 2 and
R₉ is selected from hydrogen, hydroxyl, lower alkyl, lower alkoxy, aryl, substituted aryl. hetereoaryl, substituted heteroaryl, hetereocycle or substituted heterocycle.

8. A compound of claim 1 selected from the group consisting of
rac-(2R,3R,4R,5S)-4-(4-bromo-thiophen-2-yl)-3-(3-chloro-phenyl)-4-cyano-5-(2,2-dimethyl-propyl)-pyrrolidine-2-carboxylic acid ((S)-3,4-dihydroxy-butyl)-amide,
rac-(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carboxylic acid ((S)-3,4-dihydroxy-butyl)-amide,
rac-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide,
chiral-(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carboxylic acid ((S)-3,4-dihydroxy-butyl)-amide,
rac-(2R,3S,4S,5S)-3-(3-chloro-2-fluoro-phenyl)-4-(5-chloro-3-fluoro-pyridin-2yl)-4-cyano-5-(2,2-dimethyl-propyl)-pyrrolidine-2-carboxylic acid ((S)-3,4-dihydroxy-butyl)-amide,
rac-(2R,3S,4S,5S)-4-(5-bromo-pyrimidin-2-yl)-3-(3-chloro-2-fluoro-phenyl)-4-cyano-5(2,2-dimethyl-propyl)-pyrrolidine-2-carboxylic acid ((S)-3,4-dihydroxy-butyl)-amide,
rac-4-{[(2R,3S,4S,5S)-3-(3-chloro-2-fluoro-phenyl)-4-(5-chloro-pyridin-2yl)-4-cyano-5-(2,2-dimethyl-propyl)-pyrrolidine-2-carbonyl]-amino}-benzoic acid methyl ester,
rac-4-{[(2R,3S,4S,5S)-3-(3-chloro-2-fluoro-phenyl)-4-(5-chloro-pyridin-2-yl)-4-cyano-5-(2,2-dimethyl-propyl)-pyrrolidine-2-carbonyl]-amino}-benzoic acid,
rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]amino}-benzoic acid methyl ester,
rac-4-{[(2R,3S,4S,5S)-4-(5-bromo-pyridin-2-yl)-3-(3-chloro-2-fluoro-phenyl)-4-cyano-5-(2,2-dimethyl-propyl)-pyrrolidine-2-carbonyl]-amino}-benzoic acid,
rac-(2R,3R,4R,5S)-3-(3-chloro-phenyl)-4-cyano-5-(2,2-dimethyl-propyl)-4-pyridin-3-yl-pyrrolidine-2-carboxylic acid ((S)-3,4-dihydroxy-butyl)-amide,
rac-(2R,3R,4R,5S)-3-(3-chloro-phenyl)-4-(6-chloro-pyridin-3-yl)-4-cyano-5-(2,2-dimethyl-propyl-pyrrolidine-2-carboxylic acid [2-((S)-22-dimethyl-[1,3]dioxolan-4-yl)-ethyl]-amide,
rac-(2R,3R,4R,5S)-3-(3-chloro-phenyl)-4-(6-chloro-pyridin-3-yl)-4-cyano-5-(2,2-dimethyl-propyl)-pyrrolidine-2-carboxylic acid ((S)-3,4-dihydroxy-butyl)-amide,
rac-4-{[(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carbonyl]amino}-3-methoxy-benzoic acid methyl ester
and
rac-4-{[(2R,3S,4S,5S)-3-(3-chloro-2-fluoro-phenyl)-4-(5-chloro-3-fluoro-pyridin-2-yl)-4-cyano-5-(2,2-dimethyl-propyl)-pyrrolidine-2-carbonyl]-amino}-3-fluoro-benzoic acid methyl ester.

9. A pharmaceutical formulation comprising a compound of the formula 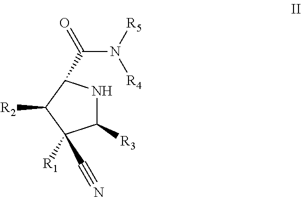
wherein
R₁ is a substituted or unsubstituted heteroaryl selected from 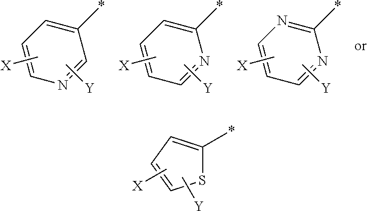
X is selected from the group consisting of H, F, Cl, Br and I
Y is H or F
R₂ is selected from the group consisting of aryl, substituted aryl, heteroaryl and substituted heteroaryl,
R₃ is selected from the group consisting of lower alkyl, substituted lower alkyl, lower alkenyl, substituted lower alkenyl, aryl, substituted aryl, heteroaryl, substituted heteroaryl, heterocycle, substituted heterocycle, cycloalkyl, substituted cycloalkyl, cycloalkenyl, and substituted cycloalkenyl,
R₄ and R₅ are selected from the group consisting of (CH₂)ₙ—R′, (CH₂)ₙ—NR′R″, (CH₂)ₙ—NR′COR″, (CH₂)ₙ—NR′SO₂R″, (CH₂)ₙ—COOH, (CH₂)ₙ—COOR′, (CH₂)ₙ—CONR′R″, (CH₂)ₙ—OR′, (CH₂)ₙ—SR′, (CH₂)ₙ—SOR′, (CH₂)ₙ—SO₂R′, (CH₂)ₙ—COR′, (CH₂)ₙ—SO₃H, (CH₂)ₙ—SONR′R″, (CH₂)ₙ—SO₂NR′R″, (CH₂CH₂O)ₘ—(CH₂)ₙ—R′, (CH₂CH₂O)ₘ—(CH₂)ₙ—OH, (CH₂CH₂O)ₘ—(CH₂)ₙ—OR′, (CH₂CH₂O)ₘ—(CH₂)ₙ—NR′R″, (CH₂CH₂O)ₘ—(CH₂)ₙ—NR′COR″, (CH₂CH₂O)ₘ—(CH₂)ₙ—NR′SO₂R″, (CH₂CH₂O)ₘ—(CH₂)ₙ—COOH, (CH₂CH₂O)ₘ—(CH₂)ₙ—COOR′, (CH₂CH₂O)ₘ—(CH₂)ₙ—CONR′R″, (CH₂CH₂O)ₘ—(CH₂)ₙ—SO₂R′, (CH₂CH₂O)ₘ—(CH₂)ₙ—COR′, (CH₂CH₂O)ₘ—(CH₂)ₙ—SONR′R″, (CH₂CH₂O)ₘ—(CH₂)ₙ—SO₂NR′R″, (CH₂)ₚ—(CH₂CH₂O)ₘ—(CH₂)ₙ—R′, (CH₂)ₚ—(CH₂CH₂O)ₘ—(CH₂)ₙ—OH, (CH₂)ₚ—(CH₂CH₂O)ₘ—(CH₂)ₙ—OR′, (CH₂)ₚ—(CH₂CH₂O)ₘ—(CH₂)ₙ—NR′R″, (CH₂)ₚ—(CH₂CH₂O)ₘ—(CH₂)ₙ—NR′COR″, (CH₂)ₚ—(CH₂CH₂O)ₘ—(CH₂)ₙ—NR′SO₂R″, (CH₂)ₚ—(CH₂CH₂O)ₘ—(CH₂)ₙ—COOH, (CH₂)ₚ—(CH₂CH₂O)ₘ—(CH₂)ₙ—COOR′, (CH₂)ₚ—(CH₂CH₂O)ₘ—(CH₂)ₙ—CONR′R″, (CH₂)ₚ—(CH₂CH₂O)ₘ—(CH₂)ₙ—SO₂R′, (CH₂)ₚ—(CH₂CH₂O)ₘ—(CH₂)ₙ—COR′, (CH₂)ₚ—(CH₂CH₂O)ₘ—(CH₂)ₙ—SONR′R″, (CH₂)ₚ—(CH₂CH₂O)ₘ—(CH₂)ₙ—SO₂NR′R″, R′ and R″ are independently selected from H, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, hetereoaryl, substituted hetereoaryl, hetereocycle, or substituted hetereocycle,
and in the case of R′ and R″ may independently link to form a cyclic structure selected from substituted or unsubstituted cycloalkyl, substituted or unsubstituted cycloalkenyl, substituted or unsubstituted heteroaryl or substituted or unsubstituted heterocycle,
m, n and p are independently 0 to 6
and the pharmaceutically acceptable salts and esters thereof together with a pharmaceutically acceptable excipient and/or carrier.
